(19) United States
(12) Patent Application Publication
(10) Pub. No.: US 2026/0325071 A1
(43) Pub. Date: Oct. 22, 2026

(54) WEARABLE VITAL-SIGNS-MONITORING APPARATUS

(71) Applicant: RHEO AG, Zurich (CH)

(72) Inventor: Philipp VETTER, Zurich (CH)

(21) Appl. No.: 19/123,023

(22) PCT Filed: Sep. 4, 2023
(86) PCT No.: PCT/EP2023/074106

§ 371 Date: Apr. 21, 2025

(30) Foreign Application Priority Data
Oct. 24, 2022 (WO) PCT/EP2022/079512

(51) Int. Cl. A61B 5/00

(52) U.S. Cl. CPC A61B 5/6824; A61B 5/6831

(57) ABSTRACT

The wearable vital-signs-monitoring apparatus comprises a vital-signs-monitoring device (6) having a housing (10) and monitoring electronics (42-48) arranged in said housing (10). It further comprises an attachment tube (8) of an elastic material forming a closed, continuous loop. The attachment tube (8) is configured to be elastically expanded to be worn on a user's limb with the device (6) being placed between the tube (8) and the limb. The device (6) has a housing (10) with a substantially convex outer surface with a raised section (32) for receiving one or more sensors (34, 36).

### TECHNICAL FIELD

The invention relates to a wearable vital-signs-monitoring apparatus and a vital-signs-monitoring device. It also relates to a kit comprising such an apparatus and to a method of use of such an apparatus.

### BACKGROUND ART

Wearable solutions for vital-signs-monitoring are typically based on placing a device, such as a smartwatch or a dedicated device, against a body part, in particular a human limb.

Such devices are e.g. based on optical, acoustic, and/or electrical measurements and are able to detect vital parameters, such as blood pressure, heart beat, body temperature, oxygen saturation, and/or breathing rate. Suitable technologies are e.g. described in WO 2016/138965A1 and WO2011/120973A1.

In operation, such devices can be cumbersome to use and expensive, which limits their application to certain scenarios only.

### DISCLOSURE OF THE INVENTION

The problem to be solved by the present invention is to provide an apparatus or device of this type that is easy to use in a large number of scenarios.

This problem is solved by the subject of the independent claims.

Hence, in a first aspect, the invention relates to a wearable vital-signs-monitoring apparatus that comprises at least the following elements:
A vital-signs-monitoring device: This is the actual hardware performing at least major parts of the measurement. It has a housing and monitoring electronics arranged within the housing.
An attachment tube of an elastic material: This tube is adapted and structured to push the device against a human limb. It forms a closed loop. In particular, the tube is a continuous loop. In this context, a “continuous loop” is to be understood such that the fabric, web, or other material of the attachment tube extends in a loop without breaks, locks, or fasteners. The tube is dimensioned to be wrap around a human limb, in particular a human arm, under elastic extension such that the device can be held between the tube and the limb. In order to fulfill this function, the tube is dimensioned to fulfill the following two criteria when it is brought into an annular (i.e. rotationally symmetric) configuration of the loop:
a) Without elastic deformation, the loop has an inner maximum diameter of less than 8 cm. In other words, it needs to be elastically expanded to reach a larger diameter.
b) Under elastic deformation, the inner diameter of the loop can be elastically expanded by at least 2 cm, in particular by at least 4 cm.

The design with a continuous loop and with an inner diameter of that can be elastically expanded by at least 4 cm allows to place the tube over a human limb and to insert the device between the tube and the limb, thereby holding it, without the need to operate any locks, hooking mechanisms, adhesives, etc.

Advantageously, the attachment tube comprises an elastic net forming the loop. Such a net is comfortable to wear over extended periods.

In an advantageous embodiment, the device can be devoid of any active adhesive arranged at the outer surface of the housing. In this context, an “active adhesive” is an adhesive that is in its sticky state. Since the device can be inserted between the limb and the tube, no such adhesive is required. The lack of active adhesive allows to more easily clean and, in particular, sterilize the device.

In another aspect, the present invention relates to a vital-signs-monitoring device that comprises at least the following elements:
Monitoring electronics. These electronics typically comprise circuitry and sensors for performing the actual measurements.
A housing for the monitoring electronics. The housing protects the electronics and provides the outer surface of the device.

According to this second aspect, the outer surface of the housing is closed and
a) convex everywhere or
b) convex except in concave locations wherein a curvature radius in any of said concave locations is at least 1 cm.

This type of housing is easy to clean and, in particular, to sterilize because the housing lacks any nooks, holes or hard-to-reach recesses at its outer surface.

In this context, “convex” also includes a flat outer surface section.

Concave-convex surface sections (i.e. saddle-shaped surface sections) are counted as convex surface sections, and any concave curvature direction of such surface sections need to have a curvature radius of at least 1 cm.

Advantageously, the outer surface is convex everywhere.

The invention also relates to a wearable vital-signs-monitoring apparatus as described above having a wearable vital-signs-monitoring device of this type.

Further, the invention relates to a kit comprising an apparatus of this type with a plurality of the attachment tubes. This allows reusing the device with different tubes.

Finally, the invention also relates to the use of such an apparatus.

This use comprises at least the following steps:
Positioning the attachment tube around a limb; and
Placing the monitoring device between the attachment tube and the limb, thereby pushing the monitoring device against a limb.

### BRIEF DESCRIPTION OF THE DRAWINGS

The invention will be better understood and objects other than those set forth above will become apparent when consideration is given to the following de-tailed description thereof. Such description makes reference to the annexed drawings, wherein:

FIG. 1 shows an embodiment of the apparatus as worn around an arm,

FIG. 2 shows a side view of a first embodiment of the device,

FIG. 3 shows a top view of the device of FIG. 3,

FIG. 4 shows an example of an attachment tube,

FIG. 5 shows a sectional view of a second embodiment of the device,

FIG. 6 shows a bottom view of the device of FIG. 5,

FIG. 7 shows a block diagram of the device,

FIG. 8 shows a third embodiment of the tube with increased stiffness in axial direction,

FIG. 9 shows an embodiment of the tube with non-continuous loop-configuration,

FIG. 10 shows an embodiment of the tube with a placement guide for receiving the device,

FIG. 11 shows a sectional view, perpendicular to the axial direction, at the location of the device, of the apparatus with the tube of FIG. 10, and

FIG. 12 shows a sectional view, parallel to the axial direction, at the location of the device, of the apparatus of FIG. 11.

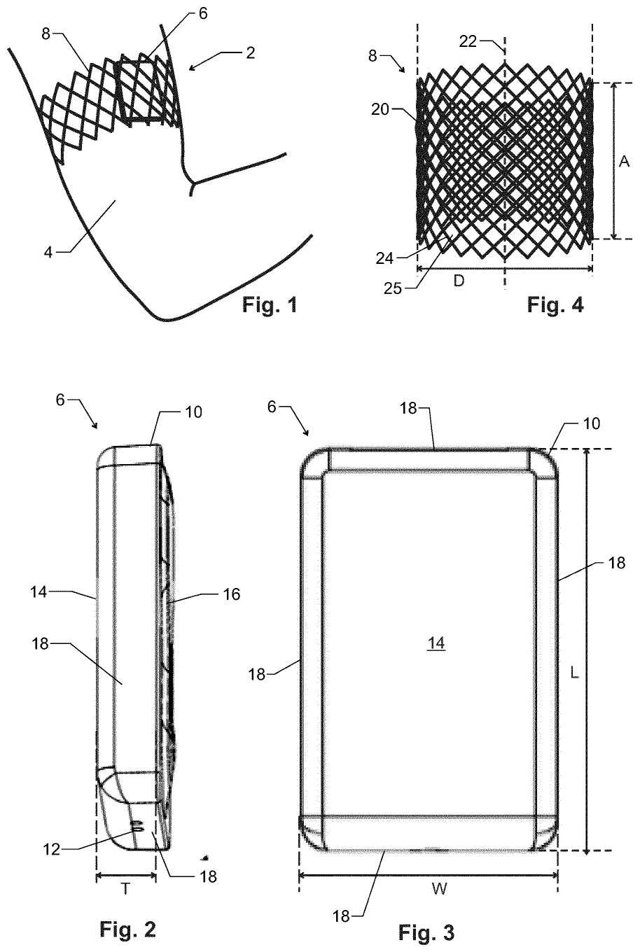

### MODES FOR CARRYING OUT THE INVENTION

### Definitions

Terms of the type top, bottom, above, and below are understood such that the (majority of) the sensors are arranged to measure through the bottom side of the device, i.e. the bottom side is the side adapted to be worn against the body when the device is in operation.

### Overview

FIG. 1 shows an embodiment of the apparatus 2 as worn on the upper arm 4 of a user. Apparatus 2 comprises a device 6 as well as an attachment tube 8. Device 6 includes the electronics and sensors for performing the measurements while attachment tube 8 is used to hold device 6 in place.

In use, device 6 is arranged between attachment tube 8 and arm 4, with attachment tube 8 pressing device 6 against the user's skin due to the elastic restoration forces within attachment tube 8.

### Device Design

Device 6 comprises a rigid housing 10. Housing 10 is advantageously of a plastic material with the possible exceptions of e.g. contact pads 12 for charging and/or one or more windows (which may e.g. be of glass) for optical sensors. Housing 10 has a top surface section 14, a bottom surface section 16 as well as a rim section 18 joining the top a bottom surface sections 14, 16 along their peripheries.

The areas of the top and bottom surface sections 14, 16 are large as compared to the area of the edge surface section 18 in order to provide a “flat” design for housing 10, which makes it easier to securely position device 6 by means of attachment tube 8 on the user's body. In particular, the area of each of the top and bottom surface section 14, 16 is at least as large as the area the rim surface section 18.

The top and bottom surface sections 14, 16 are advantageously parallel to each other, again in order to make it easier to safely mount the device between attachment tube 8 and the user's body. In this context, “parallel to each other” is to be understood within an accuracy of 15°.

The thickness T of housing 10, i.e. the distance between the top and bottom surface sections 14, 16, is advantageously in a range between 5 and 17 mm. If the device is too thin, it becomes difficult to generate a sufficient force against the body by means of attachment sleeve 8 while maintaining a comfortable wearing experience. If the device is too thick, it becomes more prone to collisions and unstable.

The width W and length L of housing 10, i.e. the shorter and longer extensions perpendicular to thickness T, are advantageously smaller than 5 cm and 8 cm, respectively, in order to place the device flushly against the user's limb.

### Attachment Tube Design

Attachment tube 8 forms a sleeve, i.e. it is of a flexible material dimensioned to loop around a limb. An embodiment is shown in FIG. 4.

The shown embodiment is formed by an elastic net 20 that loops around a central axis 22. Advantageously, it has a rotationally symmetrical design along central axis 22, i.e. the net 20 can be brought, without elastic deformation, into a cylinder configuration around axis 22 as shown.

Advantageously, the meshes of net 20 should have a diameter of at least 3 mm but no more than 25 mm in order to provide good air circulation yet still to safely hold device 6.

Attachment tube 8 should be suited to be slid over a user's limb, where the elastic compression generated by attachment tube 8 can hold device 6 in place. Hence, when tube 8 is brought into a rotationally symmetric, i.e. annular, configuration around axis 22 without being elastically extended, its inner maximum diameter D should advantageously be less than 8 cm, in particular less than 6 cm. On the other hand, under elastic deformation, this inner diameter D (again, while maintaining an annular configuration of tube 8 around axis 22), should be extendible by at least 4 cm, in particular by at least 8 cm.

In particular, attachment tube 8 is advantageously elastic enough to be elastically stretched, in linear direction, by at least a factor 5. This makes the force it generates less dependent on the actual diameter of the limb.

The axial length A of tube 8, without elastic deformation and in annular configuration, should be at least 4 cm, in particular at least 6 cm in order to safely hold device 6 against the limb

Elastic net 20 is formed by interconnected elastic strings 24. These strings 24 advantageously have a polymeric core to give them their elastic properties and a sufficiently loose textile coating to prevent the core from contacting skin. The strings 24 form elastic material sections surrounding openings 25 of the net.

For being comfortable to be worn over extended periods of time, attachment tube 8 covers only a small percentage of the skin beneath it, at least when in its extended (operative) state. Specifically, attachment tube 8 has, when elastically linearly extended by 20% along its circumferential direction, a surface ratio between its openings 25 and its material sections 24 of at least 1:1, in particular of at least 4:1. In that case, it covers less than 50%, in particular less than 20% of the skin beneath it. This not only reduces sweat but also skin irritation, and it makes tube 8 cheaper and easier to dispose after use.

In one embodiment, tube 8 can be a suitably cut section of an elastic compression net, such as a section of RETELAST ARM by Medinet SRL, Milan (IT).

As mentioned, attachment tube 8 lacks any lock and fastener. In other words, the loop formed by attachment tube 8 cannot be reversibly opened and closed. This adds to the simplicity and ease of use of the apparatus while keeping it inexpensive.

### Device Housing

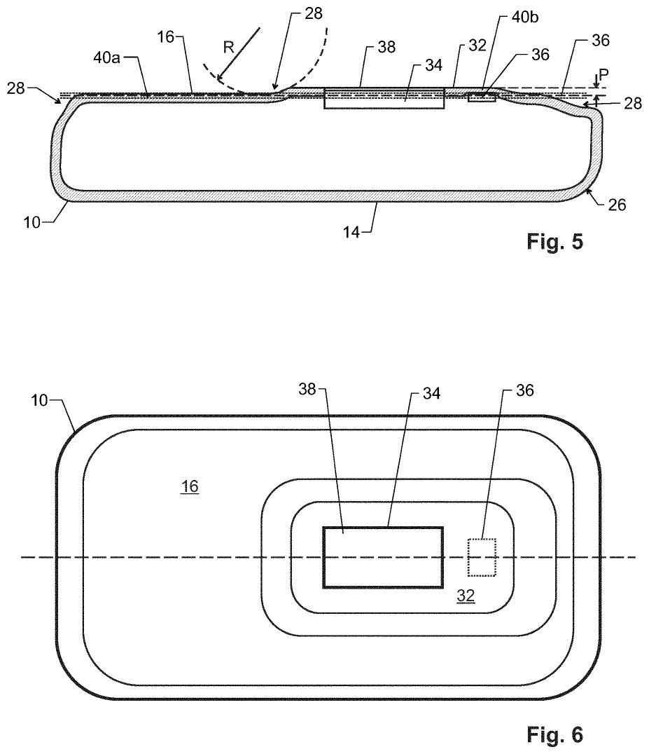

Some aspects of the design of housing 10 are now described in reference to FIGS. 5 and 6.

As mentioned above, housing 10 should either have a fully convex outer surface or an outer surface where any concave section has a curvature radius of at least 1 cm.

In the embodiment of FIGS. 5 and 6, for example, the outer surface 26 of housing 10 is mostly convex with the exception of a few concave sections 28.

Any such concave section 28, however, is only weakly curved, with its concave curvature radius R being larger than 1 cm. As a result, the outer surface 26 is housing 10 can be cleaned and in particular sterilized easily.

FIGS. 4 and 5 show another important aspect of the present design. Bottom surface section 16 (which is located at the upper side in FIG. 5), i.e. the surface section designed to face the body, extends over an area of at least 4 cm² in order to provide a stable foothold on the user's body. Most of bottom surface section 16 is flat and lies in a plane 30 within an accuracy of +2 mm except for an outwards-projecting raised section 32, which has an area of less than 2cm². Advantageously, raised section 32 projects over plane 30 by a height P of more than 0.5 mm but no more than 3 mm.

Device 6 comprises at least one sensor 34, 36 located in raised section 32. This design pushes this at least one sensor into the user's body, thereby providing more accurate measurements of the body parameters.

By way of example, device 6 comprises two such sensors 34, 36.

Sensor 34 is an optical sensor comprising e.g. a light source and light detector in order to measure the reflectivity at one or more wavelengths. Sensor 36 is a temperature sensor.

A transparent window 38 of optical sensor 24 forms part of the outer surface of housing 10.

The thickness of housing 10 may be thinned in at least part of raised section 32 to bring the respective sensor 34, 36 into even closer contact with the user's body. This is illustrated for temperature sensor 36 in FIG. 4. As can be seen, housing 10 has a first wall section 40a in bottom surface section 16 but outside raised section 32 and a second wall section 40b inside raised section 32 and covering sensor 36. Second wall section 40b is thinner than first wall section 40a, in particular by at least 50%.

In more general terms, in an advantageous embodiment, device 6 comprises a temperature sensor 36. Further, in the bottom surface section 16, housing 10 has a first wall section 40a and a second wall section 40b, with the first wall section 40a surrounding the second wall section 40b. The second wall section 40b, which is advantageously raised in respect to the first wall section, is thinner, in particular by at least 50%, than the first wall section 40 a, and the temperature sensor 36 is located within housing 10 adjacent to the second wall section 40b.

Hence, the second wall section 40b is not necessarily raised, but it may also be at the same level as the first wall section 40a (or, less advantageously, it may even be recessed). As mentioned, though, the raised arrangement of the second wall section in respect to the first wall section is advantageous.

### Device Circuitry

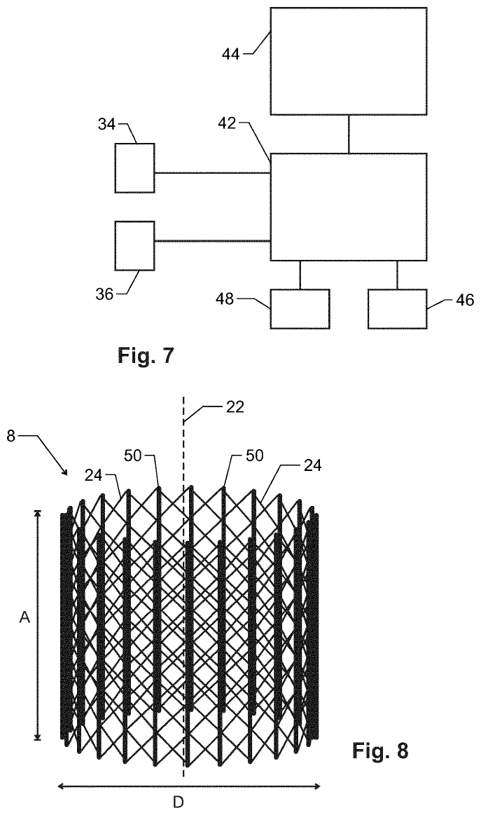

FIG. 7 illustrates an example of the circuitry in device 6.

This circuitry comprises a control unit 42, such as a microprocessor operating under control of programming stored in a memory unit 44. Control unit 42 communicates with the sensors 34, 36 of the device in order to measure body parameters, i.e. the “vital signs” as mentioned above, such as blood pressure, heart beat, body temperature, oxygen saturation, and/or breathing rate.

For example, blood pressure, heart beat frequency, oxygen saturation, and breathing rate may be measured by means of optical reflection measurements carried out by optical sensor 34 at specific wavelengths using the technologies in the documents mentioned above. Body temperature can be estimated by means of temperature sensor 36.

The circuitry of device 6 further comprises a battery 46 with a wired or wireless power adapter as well as an interface 48 for transmitting the measured parameters to an external system. Interface 48 may be wired or wireless.

### Device Utilization

To use the apparatus, attachment tube 8 is slid over the user's arm, and device 6 is inserted between tube 8 and the arm, with bottom side 16 facing the arm. In this position, tube 8 is extended and exerts an elastic force against device 6, holding it in place.

In this way, the apparatus can be worn over extended periods of time, such as for several days. It can be easily removed, e.g. for washing, and then reinstalled by the user.

Attachment tube 8 may be replaced regularly and also when device 6 is transferred to another user.

Between users, device 8 is cleaned, with the convex or nearly convex outer surface of its housing making such cleaning easy.

The apparatus can be used to monitor vital signs of hospital patients but also of persons outside a hospital, e.g. of persons in need of increased health monitoring.

Device 6 can be used over longer periods of time while attachment tube 8 has to be replaced regularly. Hence, the apparatus may advantageously be offered as a kit comprising a device 6 as well as a plurality of the attachment tubes 8. For example, the plurality of attachment tubes 8 may be included as individual sections of tube, or they may be included as a longer tube to be cut into sections of suitable length when being used.

In operation, device 6 is advantageously covered, on its surface facing away from the limb, by tube 8. The device surface facing the limb is in physical contact with the limb for better measurements.

### Attachment Tube With Increased Axial Stiffness

FIG. 8 shows an embodiment of attachment tube 8 with increased axial stiffness of compression. In particular, the axial stiffness Sa of compression along its central axis 22 is larger than the radial stiffness Sr of expansion.

The axial stiffness Sa of compression of the tube is defined as the total axial force (integrated over the whole circumference of the loop) required to compress the length A of tube 8 along central axis 22 by 10% starting from the tube in its cylindric, non-elastically deformed configuration.

The radial stiffness Sr of expansion of the tube is defined as the total radial force (integrated over the whole loop surface) required to radially and homogeneously expand the diameter D by 10% starting from the tube in its cylindric, non-elastically deformed configuration.

Advantageously, the axial stiffness Sa of compression is at least 10 times larger than the radial stiffness Sr of expansion.

This design of tube 8 has the advantage that it becomes easier to slide the tube onto a limb because it will expand easily in radial direction but be less prone to crumpling in axial direction.

In the shown embodiment, tube 8 comprises a plurality of rods 50 extending parallel to central axis 22. Adjacent rods 50 are interconnected by means of the elastic strings 24, advantageously by means of the elastic strings 24 only.

The rods 50 have a compressive stiffness (along their axial directions) that is larger than the tangential stiffness of expansion of the web of elastic strings 24.

Hence, in more general terms, tube 8 advantageously comprises a plurality, in particular at least six, rods 50 extending along the axial direction.

Adjacent rods are interconnected by a web, in particular a web of strings 24. The strings are advantageously elastic to allow expansion of the inner diameter D as defined above.

Advantageously, there are no rods extending transversally to the rods 50 since such rods might affect the radial elasticity of tube 8.

In general, it must be noted that the geometric considerations of the tube mentioned in the previous sections advantageously also apply to the embodiment of FIG. 8. In particular, when tube 8 is brought into a rotationally symmetric, i.e., annular, configuration around axis 22 without being elastically extended, its inner maximum diameter D should advantageously be less than 8 cm, in particular less than 6 cm. On the other hand, under elastic deformation, this inner diameter D (again, while maintaining an annular configuration of tube 8 around axis 22), should be extendible by at least 4 cm, in particular by at least 8 cm.

Advantageously, the rods 50 are of a plastic material, with each rod having a smallest diameter of at least 0.5 mm and a largest diameter of no more than 5 mm in order to be sufficiently stiff without being overly bulky.

### Non-Continuous Loops

As mentioned above, attachment tube 8 advantageously forms a closed, continuous loop. This makes donning it easier if the user has no one to help them.

Alternatively, though, the apparatus may also be provided with a tube variant that does not form a closed, continuous loop but rather consists of a ribbon having a lock mechanism to lock the ribbon into a closed loop.

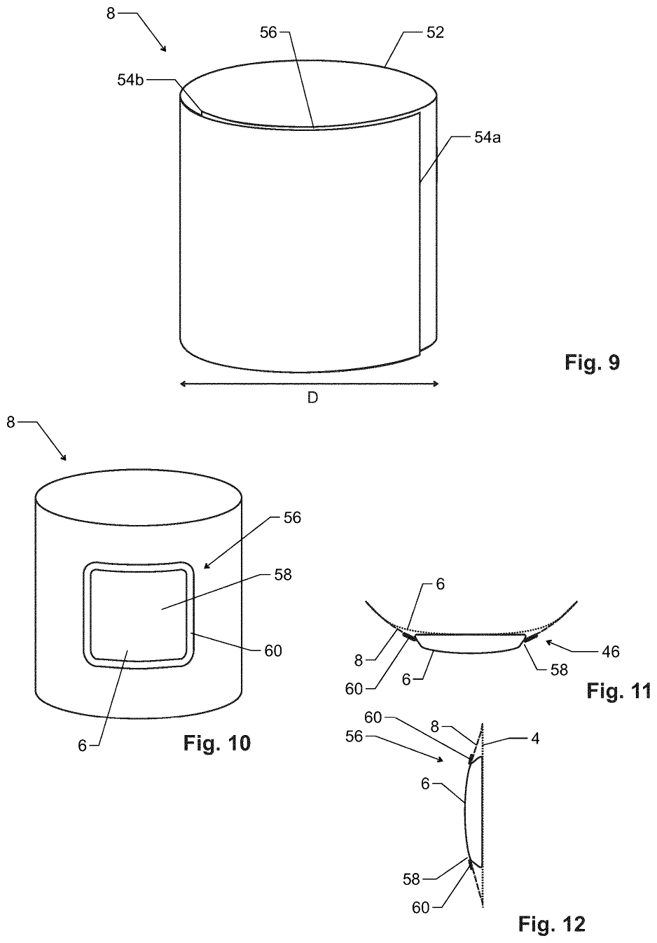

Such a variant of tube 8 is shown in FIG. 9. It comprises a ribbon 52 with first and second ends 54a, 54b. The ends can be brought into an overlapping configuration to form a loop and be attached to each other by means of the lock mechanism.

In the shown embodiment, the lock mechanism of formed by an adhesive surface 56 arranged on at least one of the ends 54a, 54b, but it may, e.g., also comprise a Velcro-type attachment, buttons, hooks, etc.

This type of tube 8 is particularly suited when the user has help in donning it.

Advantageously, the lock mechanism can be activated (locked) for different inner diameters D of the loop. This is, e.g., the case if the lock mechanism is formed by an adhesive surface or a Velcro-type attachment as mentioned above. In that case, the inner diameter D of the loop can be better adapted to the diameter of the limb. Hence, in contrast to the continuous loop embodiments above, the requirements for the elastic radial deformation of tube 8 are less stringent. In general, it may be sufficient if the inner diameter D of the loop can be elastically expanded by at least 2 cm in order to generate a sufficient force to push device 6 against the limb.

Ribbon 52 may, e.g., be formed by an elastic bandage. It may, however, also be formed by an elastic net as described above.

Hence, in more general terms, attachment tube (8) advantageously comprises at least the following parts:
A ribbon 52 with first and second ends 54a, 54b: This ribbon can be configured to form the loop.
A lock mechanism 56 adapted and structured to attach the first and second ends 54a, 54b to each other, thereby forming said loop from the ribbon 52.

### Device Placement Guide

In the embodiments above, and as illustrated by FIG. 1, device 6 is simply placed between tube 8 and the user's limb 4. This allows for a very simple tube design.

In some situations, though, a more accurate and defined placement of device 6 is desired. For example, when the apparatus is to be worn during physical exercise, such as jogging, it may be advantageous to provide means to keep device 6 in place even when strong forces are applied to it.

Hence, as shown in FIGS. 10-12 , tube 8 may be provided with a placement guide 56 for locking device 6 into place.

Advantageously, placement guide 56 comprises an opening 58 extending radially through tube 8.

In its assembled configuration, device 6 is located partially within the tube 8, with part of device 6 extending through opening 58. The circumference of opening 58 is smaller than the circumference of device 6 (i.e., in its operating position, device 6 cannot be pushed outwards to completely pass through opening 58). Hence, tube 8 is still able to push device 6 against the limb 4. But since part of device 6 extends through opening 58, its position is locked against tangential and axial displacements in respect to tube 8.

Advantageously, and as shown in FIGS. 10-12 , opening 58 is surrounded by a frame 60. Frame 60 has a higher stiffness than any tube material tangentially and axially surrounding it. This allows to firmly hold device 6 in place even if the tube material is, in at least radial direction, highly deformable.

Frame 60 may, e.g., be a frame of plastic material.

Hence, advantageously, tube 8 comprises an opening 58, wherein device 6 is located partially within tube 8, with part of device 6 extending through opening 58.

Placement guide 56 can be combined with any of the embodiments of the apparatus shown herein.

Advantageously, tube 8 comprises only a single opening 58 for receiving device 6.

### Notes

In the examples above, the apparatus is shown to be worn around the upper arm of a user. It may, however, also be worn around the lower arm or around a leg. Advantageously, attachment tubes 8 of suitable sizes are provided for such purposes.

As shown and described above and in the drawings, the sensor and electronics of the apparatus are advantageously located within device 6, with no sensors or electronics arranged in tube 8. This makes the design of tube 8 simpler and installing device 6 easier. In particular, there are no electrical connections between device 6 and tube 8.

Hence, tube 8 is advantageously of dielectric material only and comprises no metal, which makes it easier to manufacture and/or to wash.

While there are shown and described presently preferred embodiments of the invention, it is to be distinctly understood that the invention is not limited thereto but may be otherwise variously embodied and practiced within the scope of the following claims.

1. A wearable vital-signs-monitoring apparatus comprising
a vital-signs-monitoring device having a housing and monitoring electronics arranged in said housing, and
an attachment tube of an elastic material forming a closed loop, wherein, in annular configuration,
without elastic deformation, said loop has an inner maximum diameter of less than 8 cm and
under elastic deformation, the inner diameter of the loop is extendible by at least 2 cm,

2. The apparatus of claim 1 wherein, in annular configuration, without elastic deformation, said loop has an inner diameter of less than 6 cm.

3. The apparatus of claim 1 wherein, in annular configuration and under elastic deformation, the inner diameter of the loop is extendible by at least 8 cm.

4. The apparatus of claim 1 wherein said attachment tube comprises an elastic net having elastic material sections surrounding openings forming said loop.

5. The apparatus of claim 4 wherein said elastic net comprises elastic strings having a polymeric core and a textile coating.

6. The apparatus of claim 4, wherein the mesh has, when elastically linearly extended by 20%, a surface ratio between the openings and the material sections of at least 1:1,

7. The apparatus of claim 1 wherein, without elastic deformation and in annular configuration, said loop has an axial length of at least 4 cm,

8. The apparatus of claim 1 without any active adhesive arranged at an outer side of the housing

9. The apparatus of claim 1 wherein an axial stiffness of compression of the tube along a central axis of the tube is larger than a radial stiffness of expansion of the tube.

10. (canceled)

11. The apparatus of claim 1 wherein
the attachment tube forms a closed, continuous loop and
in annular configuration, under elastic deformation, the inner diameter of the loop is extendible by at least 4 cm.

12. The apparatus of claim 1 wherein the attachment tube comprises
a ribbon with first and second ends and
a lock mechanism adapted and structured to attach the first and second ends, thereby forming said loop from the ribbon.

13. The apparatus of claim 1 wherein said tube comprises an opening wherein the device is located partially within the tube with part of the device extending through the opening.

14. The apparatus of claim 13 further comprising a frame surrounding the opening with said frame having a higher stiffness than any tube material surrounding the frame.

15. The apparatus of claim 1 wherein all sensors of the apparatus are arranged in the device.

16. The apparatus of claim 1 wherein the tube is of dielectric material only.

17. A vital-signs-monitoring device comprising
monitoring electronics and
a housing for the monitoring electronics, wherein the housing has a closed outer surface that is
convex everywhere or
convex except in concave locations wherein a curvature radius in any of said concave locations is at least 1 cm.

18. The device of claim 17 wherein the outer surface is convex everywhere.

19. The device of claim 17, wherein the housing comprises a top surface section a bottom surface section and a rim surface section joining the top and bottom surface sections wherein the top and bottom surface sections are parallel to each other with an accuracy of 15°.

20. The device of claim 19 wherein an area of each of the top and bottom surface section is at least as large as an area the rim surface section.

21. The device of claim 17 wherein said device comprises a temperature sensor and
wherein, in the bottom surface section said housing has a first wall section and a second wall section with the first wall section surrounding the second wall section wherein the second wall section is thinner, in particular by at least 50%, than the first wall section and wherein the temperature sensor is adjacent to the second wall section.

22-26. (canceled)
